(19) United States
(12) Patent Application Publication
(10) Pub. No.: US 2024/0281702 A1
(43) Pub. Date: Aug. 22, 2024

(54) METHOD AND SYSTEM FOR EMISSIONS-BASED ASSET INTEGRITY MONITORING AND MAINTENANCE

(71) Applicant: FMC Technologies, Inc., Houston, TX (US)

(72) Inventors: Tommy Thammavongsa, Houston, TX (US); Matt Byrne, Houston, TX (US); Tom Gilmour, Houston, TX (US); Beau Burgin, Houston, TX (US)

(73) Assignee: FMC Technologies, Inc., Houston, TX (US)

(21) Appl. No.: 18/172,452

(22) Filed: Feb. 22, 2023

(51) Int. Cl. G06N 20/00; G16C 20/30; G16C 20/70

(52) U.S. Cl. CPC G06N 20/00; G16C 20/30; G16C 20/70

(57) ABSTRACT

A method involves obtaining current asset data for an asset, the current asset data including process data. The method further involves predicting, using a machine learning model, a methane emissions event associated with the asset, based on the current asset data, and reporting the predicted methane emissions event in a user visualization.

### BACKGROUND

Industrial emissions such as methane emissions can be problematic for various reasons. For example, regulatory requirements may limit such emissions to a certain level, society is increasingly aware of issues resulting from the release of greenhouse gases (such as methane), etc. However, in complex industrial environments it is not necessarily straightforward to anticipate unintentional releases (e.g., leaks) and/or intentional releases which may be triggered by certain production-related events. Accordingly, while challenging, it may be desirable to anticipate releases and to take action to minimize or eliminate such releases.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a method, comprising: obtaining current asset data for an asset, the current asset data comprising process data; predicting, using a machine learning model, a methane emissions event associated with the asset, based on the current asset data; and reporting the predicted methane emissions event in a user visualization.

In general, in one aspect, embodiments relate to a system, comprising: a computing environment that: obtains current asset data for an asset, the current asset data comprising process data, predicts, using a machine learning model, a methane emissions event associated with the asset, based on the current asset data; and a dashboard comprising a user visualization that reports the predicted methane emissions event.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 shows a system for emissions-based asset integrity monitoring and maintenance, in accordance with one or more embodiments.

FIG. 2 shows a system for emissions-based asset integrity monitoring and maintenance, in accordance with one or more embodiments.

FIG. 3 shows a flowchart of a method for emissions-base asset integrity monitoring and maintenance, in accordance with one or more embodiments.

FIG. 4 shows a flowchart of a method for emissions-base asset integrity monitoring and maintenance, in accordance with one or more embodiments.

FIG. 5A, 5B, 5C, and 5D show examples of a dashboard visualizations, in accordance with one or more embodiments.

FIG. 6 shows a computer system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

In general, embodiments of the disclosure include systems and methods for emissions-based asset integrity monitoring and maintenance. It may be desirable and important to detect, or preferably predict emissions, whether intentional or unintentional. Industrial equipment includes many components that may leak. For example, leaks may develop at vapor recovery units (VRUs), compressors, valves, loose flanges, failed seals, etc. Frequency-based inspection are typically scheduled to detect such leaks. However, these inspections would miss intermittent leaks. Further, even when finding a leak, cause determination may be difficult. Similarly, the way an industrial process is performed may determine whether intentional releases are necessary. With the complexity of industrial processes, adjusting all involved parameters to avoid or minimize intentional releases may be non-trivial.

Embodiments of the disclosure ingest different types of data to predict the likeliness of an event such as a leak or a release (broadly termed an “emission”) and/or to suggest possible mitigating actions.

The prediction may be performed by a machine learning model, as further discussed below. The machine learning model may have been trained using archived (i.e., historical) data associated with detected emissions events. Due to the predictive operation of the machine learning model, once trained, risky assets that are prone to leaks may be identified prior to the occurrence of the leaks. A detailed description is subsequently provided.

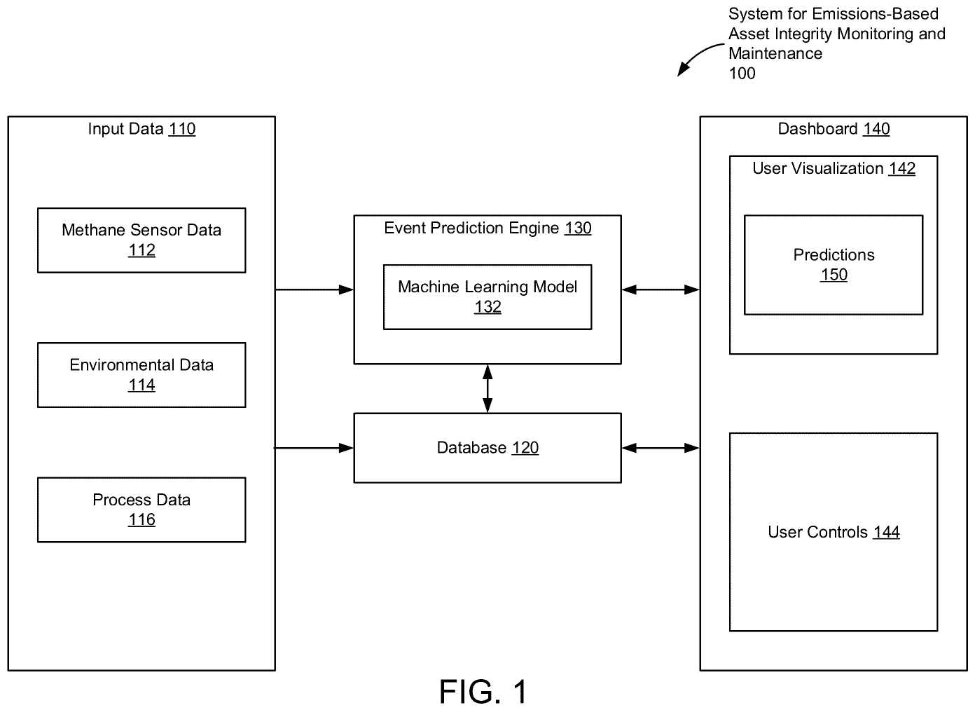

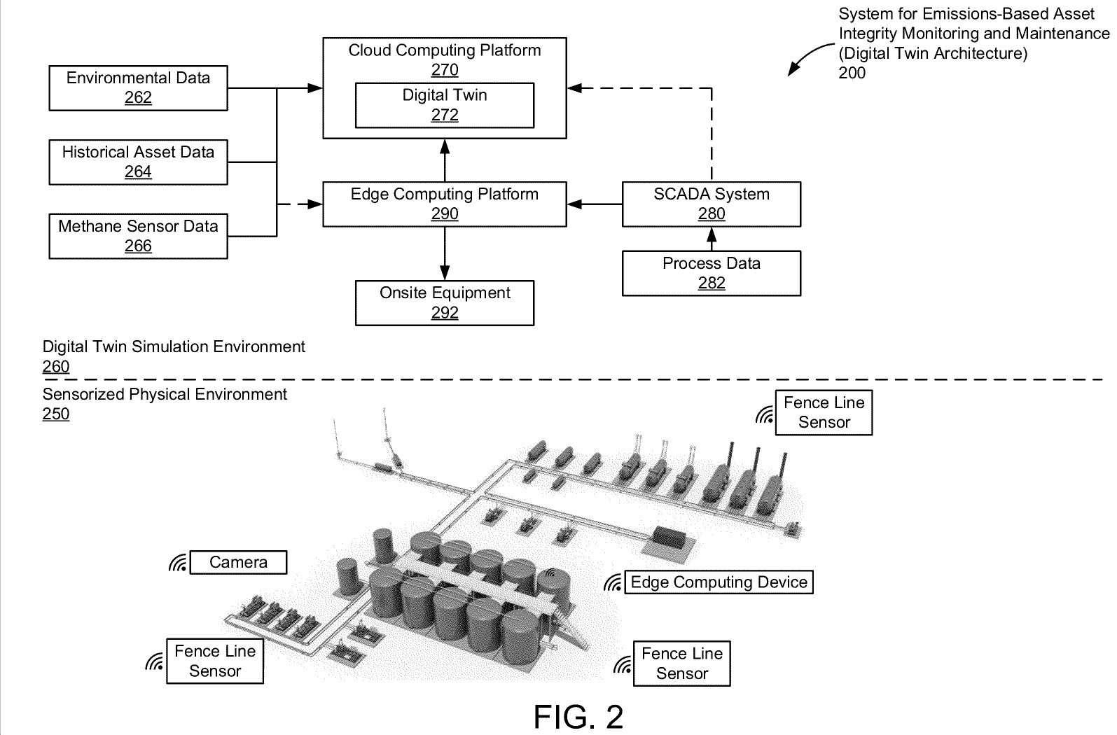

FIG. 1 shows a system for emissions-based asset integrity monitoring and maintenance, in accordance with one or more embodiments. FIG. 1 provides an overview of components of the system 100, whereas FIG. 2 provides more implementation-specific details.

The system 100 includes an event prediction engine 130 that operates on input data 110 and/or data obtained from a database 120 to output predictions 150 displayed to a user in a dashboard 140 environment. Each of these components is subsequently described.

The input data 110 may include any type of data that may be collected from one or more assets to be monitored. While the asset to be monitored may be an individual piece of equipment, more commonly multiple assets with potentially complex interactions may be monitored. An example is a petrochemical operation such as a refinery or, more generally, an industrial site. Accordingly, an asset may be a petrochemical asset. Examples for assets include, but are not limited to:
bulk separators, test separators, spools, flanges, relief valves, isolation valves, choke valves, flow meters, density meters, burn management devices, flare stacks, vapor recovery units (VRUs), vapor recovery tower (VRTs), burn management devices/systems, storage tanks, water tanks, heater treaters, air actuated valves, electric actuated valves, production trees, pump jacks, gathering lines, compressors, pumps, power units, and thief hatches on storage tanks.

Sensors may be placed in proximity (or in the general area) of the asset(s) These sensors may collect methane sensor data 112. In addition, environmental data 114 and process data 116 may be collected. These data may be collected in real-time, e.g., at sample rates of the sensors being used for the data collection. Any sample rate may be used. For example, sample rates may be in the range of milliseconds, seconds, minutes, hours, days, etc. Data may also be buffered, i.e., the data may not be real-time data.

The methane sensor data 112 may be used to detect and/or quantify emissions, e.g., methane emissions. The methane sensor data may be obtained in various different manners, including, but not limited to:
fenceline monitoring, thermal camera monitoring, non-thermal camera monitoring, optical gas imaging (OGI) camera monitoring, point sensor monitoring, drone monitoring, robot monitoring, helicopter monitoring, airplane monitoring, and satellite monitoring.

The sensors used for gathering the methane sensor data 112 may detect emissions beyond an emission level threshold, the location of an emissions event, e.g., using camera-based technology or approximation based off other deployment types, may provide an event quantification (leak rate), an event concentration (ppb or ppm), time stamps of events, and/or durations of events.

Additionally, while examples for methane detection, monitoring, and prediction are described herein, e.g., with reference to FIG. 1, methods and system disclosed herein may equally be applied for monitoring and predicting other pollutants, such as carbon monoxide or carbon dioxide. In such implementations, pollutant sensors specific to the pollutant being monitored and predicted may be used in place of or in addition to the methane sensors used to collect methane sensor data 112.

The environmental data 114 may provide additional information that may be, directly or indirectly, causally related to emission events. Environmental data may include, for example:
weather data (history and/or forecast), ambient temperature in the area, humidity, lithology, elevation, air quality, background methane readings, wind speed, and wind direction.

The process data 116 may provide information on the process that may be, directly or indirectly, causally related to emission events. Process data may be collected, for example, using sensors installed on the previously described assets. Process data may include, for example:
pressure, temperature, liquid flow, liquid level, Reid vapor pressure, flare meter readings, fluid density, vibration, acoustic patterns, voltage resistivity, electric current, valve settings, and burn management readings.

The described data may be collected in the form of time series, e.g., for each sensor reading.

Continuing with the discussion of the system 100, the database 120 may be used to accumulate archived (historical) data, e.g., methane sensor data 112, environmental data 114, process data 116, and/or other data. For example, the database may also store archived data associated with the asset, including operational data, and also economic data. Archived asset data may include an event history of assets. For example, the historical asset data may include an inspection history, failures, repairs, inspection schedules, etc., of assets. Operational data may include information related to the operation of the assets. For example, operational data may include deployment facilities in region, distance from facility to site, regional headcount, competency per headcount, availability of headcount, fleet vehicles to deploy for service, asset inventory and capacity for all facilities, approved vendor list for assets, pricing of assets from vendors, electricity usage onsite, description and serial numbers of assets onsite, condition of each asset, inspection history of assets, failure history of assets, repair history of assets, inspection schedule of assets, cellular service strength and bit rate, manufacturer of assets. Economic data may include, for example, carbon pricing, methane pricing, methane penalty fines, etc., which may affect decisions regarding intentional releases of methane.

These data may be used to train a machine learning model, as discussed below. Not all of these data are necessarily relevant for the purpose of the prediction of methane emissions events. However, through training of the machine learning model, the relevant data are identified, thereby enabling the machine learning model to predict methane emissions events.

The event prediction engine 130, in one or more embodiments, predicts methane emissions events. This may include the unintentional release of methane, e.g., through leaks and/or the intentional release of methane, e.g., through flaring. The predictions, in one or more embodiments, are made by the machine learning model 132. What is predicted by the machine learning model 132 depends on the available data provided as input data 110 and obtained from the database 120, and further on the training of the machine learning model 132. Through training, the machine learning model 132 learns to correlate input data w/methane emissions events, thereby acquiring the capability to predict such events, including magnitude and timing of these events, equipment involved, etc.

The event prediction engine may also include algorithms for performing the training of the machine learning model. A few examples of machine learning models and the training of these machine learning models are subsequently discussed. Other machine learning models of combinations of machine learning models may be used without departing from the disclosure.

Based on the availability of historical event data in the form of methane sensor data 112, documented releases, and other types of input data and database data, a supervised learning approach may be used to train the machine learning model 132 to predict methane emissions events based on current input data and database data. The machine learning model may be a classifier that may classify the received data into different classes, e.g., based on the significance of a methane emissions event. For example, a release event may be classified as minor, medium, or large, depending on the amount of methane released. The classes and thresholds associated with these classes may be customizable. For example, a major event may be an even involving the release of 5 kg/h or 10 kg/h of methane, which may be beyond limits established by regulations. The machine learning model may further predict the timing of the release and the cause of the release (e.g., a particular asset). The machine learning model may also predict possible mitigating actions. Other types of machine learning models, different from classifiers, e.g., regression models, may be used, and training methods other than supervised learning may be used, without departing from the disclosure.

The machine learning models may be based on any type of machine learning technique/algorithm. For example, perceptrons, convolutional neural networks, deep neural networks, recurrent neural networks, support vector machines, decision trees, inductive learning models, deductive learning models, supervised learning models, unsupervised learning models, reinforcement learning models, etc. may be used. In some embodiments, two or more different types of machine-learning models are integrated into a single machine-learning architecture, e.g., a machine-learning model may include support vector machines and neural networks.

In some embodiments, various types of machine learning algorithms, e.g., backpropagation algorithms, may be used to train the machine learning model. In a backpropagation algorithm, gradients are computed for each hidden layer of a neural network in reverse from the layer closest to the output layer proceeding to the layer closest to the input layer. As such, a gradient may be calculated using the transpose of the weights of a respective hidden layer based on an error function (also called a “loss function”). The error function may be based on various criteria, such as mean squared error function, a similarity function, etc., where the error function may be used as a feedback mechanism for tuning weights in the machine-learning model. In some embodiments, historical data, e.g., methane sensor data, environmental data, process data and/or database data recorded over time, may be augmented to generate synthetic data for training the machine learning model.

With respect to neural networks, for example, a neural network may include one or more hidden layers, where a hidden layer includes one or more neurons. A neuron may be a modelling node or object that is loosely patterned on a neuron of the human brain. In particular, a neuron may combine data inputs with a set of coefficients, i.e., a set of network weights for adjusting the data inputs. These network weights may amplify or reduce the value of a particular data input, thereby assigning an amount of significance to various data inputs for a task being modeled. Through machine learning, a neural network may determine which data inputs should receive greater priority in determining one or more specified outputs of the neural network. Likewise, these weighted data inputs may be summed such that this sum is communicated through a neuron's activation function to other hidden layers within the neural network. As such, the activation function may determine whether and to what extent an output of a neuron progresses to other neurons where the output may be weighted again for use as an input to the next hidden layer.

Turning to recurrent neural networks, a recurrent neural network (RNN) may perform a particular task repeatedly for multiple data elements in an input sequence (e.g., a sequence of maintenance data or inspection data), with the output of the recurrent neural network being dependent on past computations. As such, a recurrent neural network may operate with a memory or hidden cell state, which provides information for use by the current cell computation with respect to the current data input. For example, a recurrent neural network may resemble a chain-like structure of RNN cells, where different types of recurrent neural networks may have different types of repeating RNN cells. Likewise, the input sequence may be time-series data, where hidden cell states may have different values at different time steps during a prediction or training operation. For example, where a deep neural network may use different parameters at each hidden layer, a recurrent neural network may have common parameters in an RNN cell, which may be performed across multiple time steps. To train a recurrent neural network, a supervised learning algorithm such as a backpropagation algorithm may also be used. In some embodiments, the backpropagation algorithm is a backpropagation through time (BPTT) algorithm. Likewise, a BPTT algorithm may determine gradients to update various hidden layers and neurons within a recurrent neural network in a similar manner as used to train various deep neural networks. In some embodiments, a recurrent neural network is trained using a reinforcement learning algorithm such as a deep reinforcement learning algorithm. For more information on reinforcement learning algorithms, see the discussion below.

Embodiments are contemplated with different types of RNNs. For example, classic RNNs, long short-term memory (LSTM) networks, a gated recurrent unit (GRU), a stacked LSTM that includes multiple hidden LSTM layers (i.e., each LSTM layer includes multiple RNN cells), recurrent neural networks with attention (i.e., the machine-learning model may focus attention on specific elements in an input sequence), bidirectional recurrent neural networks (e.g., a machine-learning model that may be trained in both time directions simultaneously, with separate hidden layers, such as forward layers and backward layers), as well as multidimensional LSTM networks, graph recurrent neural networks, grid recurrent neural networks, etc., may be used. With regard to LSTM networks, an LSTM cell may include various output lines that carry vectors of information, e.g., from the output of one LSTM cell to the input of another LSTM cell. Thus, an LSTM cell may include multiple hidden layers as well as various pointwise operation units that perform computations such as vector addition.

In some embodiments, one or more ensemble learning methods may be used in connection to the machine-learning models. For example, an ensemble learning method may use multiple types of machine-learning models to obtain better predictive performance than available with a single machine-learning model. In some embodiments, for example, an ensemble architecture may combine multiple base models to produce a single machine-learning model. One example of an ensemble learning method is a BAGGing model (i.e., BAGGing refers to a model that performs Bootstrapping and Aggregation operations) that combines predictions from multiple neural networks to add a bias that reduces variance of a single trained neural network model. Another ensemble learning method includes a stacking method, which may involve fitting many different model types on the same data and using another machine-learning model to combine various predictions.

Continuing with the discussion of the system 100, the dashboard 140 provides user access to components of the system 100. The dashboard 140 may be provided in the form of a user interface and may include a user visualization 142 and user controls 144. The user visualization 142, in one or more embodiments, provides predictions 150 made by the machine learning model 132. These predictions may be visualized in different manners. For example, the predictions may be provided in the form of text, tables, graphs, diagrams, etc. Examples are provided below. The user controls 144 may allow a user to control the visualization of the predictions 150 in the user visualization 142. For example, the user controls 144 may allow the user to zoom and/or pan when reviewing graphically visualized predictions, to scroll through tables, to select/deselect predictions, etc. The user controls 144 may further allow a user to take corrective/preventive action by issuing commands to equipment associated with the assets to prevent and/or mitigate a predicted release event. An example for such a corrective active is the adjustment of a pressure by changing the setting of a valve to avoid a future emergency venting event. Additional details are provided below.

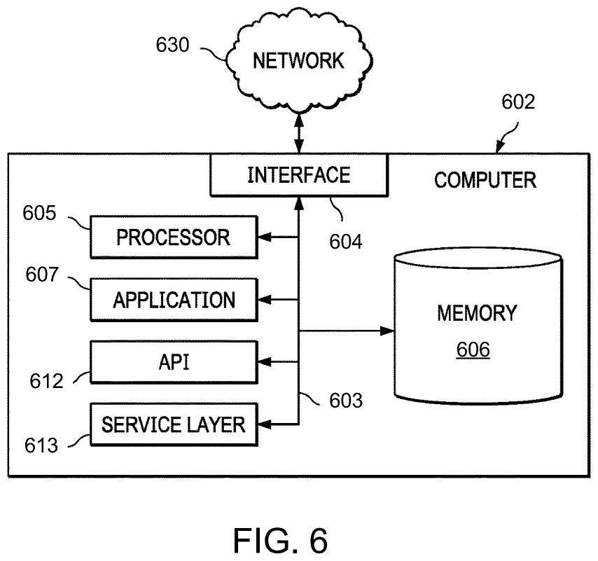

While not explicitly shown in FIG. 1, various components of the system 100 may be implemented on one or more computing systems, e.g., as shown in FIG. 6 and discussed below.

FIG. 2 shows a system for emissions-based asset integrity monitoring and maintenance, in accordance with one or more embodiments. In comparison to the system 100 in FIG. 1, additional details are shown for the system 200. FIG. 2 is separated into a lower panel showing a sensorized physical environment 250 and an upper panel showing a digital twin simulation environment 260.

In one embodiment the sensorized physical environment 250 is a production site and includes various assets such as storage tanks, power units, other assets listed herein, etc. The various assets on the production site may be used, for example, to run a complex petrochemical process, e.g., refining. The physical environment is sensorized with various sensors, including three fenceline sensors, and a camera-based sensor. These sensors may be configured to monitor methane emissions. Any number of sensors for any types of emissions may be used, without departing from the disclosure. The sensors are configured to wirelessly communicate with an edge computing device to provide their methane sensor data, e.g., at a set sample rate.

In one or more embodiments, the digital twin simulation environment 260 establishes a virtual model that reflects characteristics of the physical environment 250, i.e., a digital twin 272. In the example of FIG. 2, the digital twin 272 may primarily reflect characteristics of the physical environment that are directly or indirectly related to methane emissions. Other characteristics may also be reflected by the digital twin 272. In the example of FIG. 2, the digital twin 272 is executed on a cloud platform 270. Alternatively, the digital twin may be executed elsewhere, e.g., locally. The digital twin 272 may be partially or entirely based on the previously introduced machine learning model 132. The cloud computing platform 270 forms a central node of the digital twin simulation environment 260 that is capable of ingesting input data provided in many different forms and may process these data to be in a format suitable for the digital twin 272. For example, methane emission measurements may be provided by the camera in the sensorized physical environment 250 as an image, whereas the fenceline sensors may report a concentration. In either case, the methane emissions data, although in completely different form, once processed, may be provided to the digital twin in a format such as: timing (time stamp, start and stop), quantification, location, and facility. Additional processing may be performed, for example to eliminate false positives. As a result of the processing performed by the cloud computing platform 270, the digital twin 272 may be sensor agnostic.

The digital twin 272 may operate on various input such as environmental data 262, historical asset data 264, methane sensor data 266, process data 282, and/or other data, as previously discussed. Based on these inputs, the digital twin 272 may simulate the state of assets in the physical environment. The digital twin may also be used to simulate the state of assets into the future. In other words, the digital twin may be used in a predictive manner, for example, to predict future methane emissions.

The digital twin simulation environment 260 further includes an edge computing platform 290. The edge computing platform 290, in one or more embodiments, collects data (e.g., process data 282 accessible via a supervisor control and data acquisition (SCADA) system 280) and transfers these data to the cloud computing platform 270, and further executes control logic to operate onsite equipment 292. The edge computing platform may, thus, enable implementation of mitigating action when a methane emissions event is predicted, e.g., by operating a valve or by making other adjustments to the process in the physical environment. In a specific example, a high facility inlet temperature is detected. The elevated temperature, without proper mitigation, could result in tank venting. However, this condition and the possible consequence may have been learned by the machine learning model from archived data. In addition, the learning may also cover previously used actions to address the condition, such as (i) turning on a cooler upstream of the tanks; (ii) lowering of the VRT pressure; and (iii) lowering of the inlet vessel pressure. Accordingly, with one or more of these mitigating actions recommended by the machine learning model, the venting of the tank may not be necessary. While a single example is provided, with proper training, other associations may be made between input data, possible emissions events, and mitigating actions. The edge computing platform 290 and optionally the cloud computing platform 270 may also receive service data, e.g., entered in the SCADA system 280 in the form of field reports, e.g., by a leak detection and repair (LDAR) crew.

While not explicitly shown, various components of the system 200 of FIG. 2 may be implemented on one or more computer systems, e.g., as shown in FIG. 6 and described below. Further, while FIGS. 1 and 2 shows various configurations of components, other configurations may be used without departing from the scope of the disclosure. For example, various components in FIGS. 1 and 2 may be combined to create a single component. As another example, the functionality performed by a single component may be performed by two or more components.

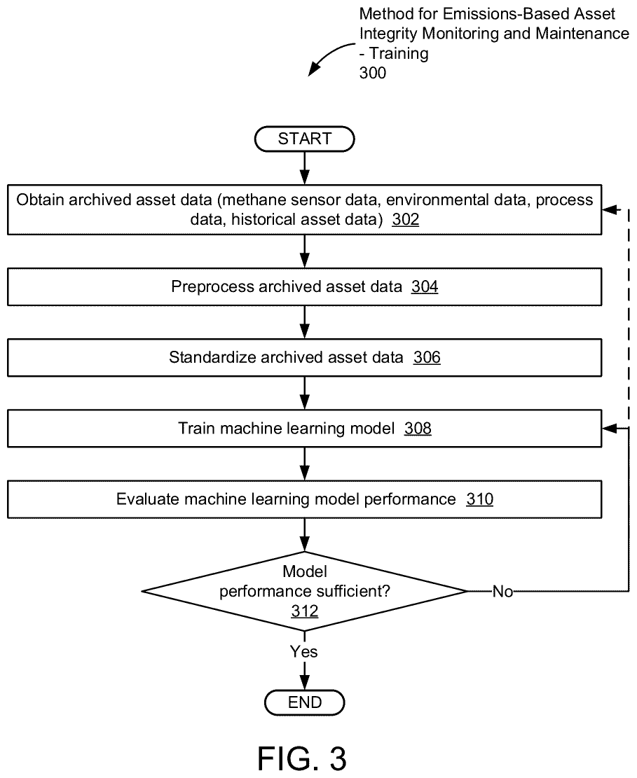

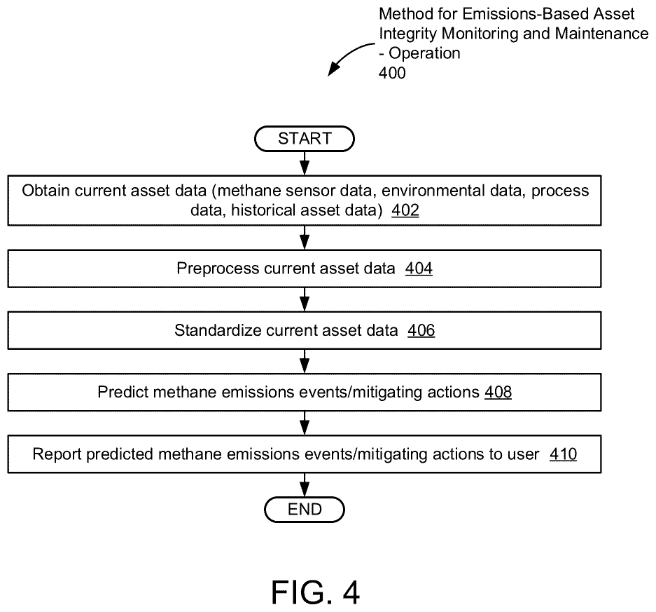

FIGS. 3 and 4 show flowcharts in accordance with one or more embodiments. One or more blocks in FIG. 3 may be performed by one or more components (e.g., event prediction engine 130 as described in FIG. 1). While the various blocks in FIG. 3 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

Turning to FIG. 3, a method 300 for training a machine learning model, in accordance with one or more embodiments, is shown. The machine learning model may be trained to predict emissions events, to predict preventive actions, etc. Once trained, the machine learning model may be used to perform predictions, as discussed below in reference to FIG. 4.

In Step 302, archived asset data are obtained. The archived asset data may include methane sensor data, environmental data, process data and/or historical asset data, as previously described. The archived asset data may be obtained from a database and may include data for the asset operating over at certain time interval, e.g. day, weeks, years, etc. The time interval may be selected such that sufficient archived asset data are available for performing the training of the machine learning model, discussed below.

In Step 304, the archived asset data are preprocessed. Outliers in the archived asset data may be detected and removed. Moving windows and/or z-score techniques may be used for the outlier detection. Further, false positives may be removed. False positives may be removed using thresholding or using human domain expertise. For example, an elevated methane concentration may be reported when a truck leaking methane is driving past a sensor. In this case, the elevated methane concentration would be a false positive. The false positive may be eliminated because the concentration is sub-threshold, because the elevated concentration is registered only for a brief moment, and/or because a field operator annotated the methane data to indicate the false positive.

In Step 306, the archived asset data are standardized. The standardizing may involve any steps required to transform elements of the asset data to form a feature vector at the input of the machine learning model, and further a representation of the output of the machine learning model. The standardization may enable a sensor agnostic operation of the machine learning model. For example, methane emission measurements may be provided by a camera as an image, whereas fenceline sensors may report a concentration. In either case, the methane emissions data, although in completely different form, once standardized may be in the same format such as: timing (time stamp, start and stop), quantification, location, and facility. The processing performed in Step 306 may attempt to extract the maximum of information from the methane sensor data. For example, camera data may be processed to estimate methane emissions based on a plume visible in the image frames, but in addition other information such as the type of visible equipment in the image frames may be extracted as well.

In Step 308, a machine learning model for prediction of methane emissions events and/or possible mitigating actions is trained. The machine learning model may be a single machine learning models making all predictions, or it may consist of multiple machine learning models that are separately trained. For example, one machine learning model may be trained using training data suitable for the prediction of methane emissions events, and another machine learning model may be trained using training data suitable for the prediction of mitigating actions. Also, separate machine learning models may or may not be used for separate assets of the physical environment being monitored. The training may be performed as previous described.

The training may further involve selecting the features to be considered by the machine learning model. Initially, all archived data may be considered by the machine learning model. However, the machine learning model may later be retrained with a reduced number of features. Specifically, some features may be identified as less relevant in comparison to other features. For example, analysis of the archived data may show that the environmental variable “wind direction” has no effect on methane emissions events, whereas the environmental variable “ambient temperature” may have a strong effect on methane emissions events. In this case, it may be beneficial to eliminate “wind direction” from the feature set. Accordingly, the less relevant features may be excluded when retraining the machine learning model.

Further, when performing the training, expert information may be incorporated into the machine learning model in order to optimize the data weighting. Expert information may include, for example, user-provided information such as known methane leak issues of a certain type of assets after a number of years of operation, known variables that strongly affect the likeliness of methane emissions, etc.

Once trained, the output of the machine learning model is an estimate of methane emissions events. The predicted information may depend on the training. For example, the machine learning model may or may not be trained to predict the magnitude of the predicted emissions (e.g., in the form of classes from smaller to larger emissions) the timing of the predicted emissions, the likely cause of the predicted emissions, etc. Similarly, whether the machine learning model predicts mitigating actions depends on whether the historical data used for training included episodes of mitigating actions to prevent or address methane emissions events.

In Step 310, the performance of the machine learning model is evaluated. The evaluation may be performed by applying the trained machine learning model to a volume of test data. The resulting estimates of methane emissions events and/or mitigating actions may be compared to actual methane emissions events and/or mitigating actions in the test data. In one or more embodiments, evaluating the performance of the machine learning model further includes evaluating the feature set of the machine learning model. Each feature in the feature set may be analyzed on its impact on the estimate produced by the machine learning model. The impact may be assessed, for example, using Shapley values. As previously noted, features with little to no impact may be eliminated from the feature set.

In Step 312, a decision is made regarding the sufficiency of the machine learning model accuracy. Whether the model performance is sufficient may be determined based on the degree of deviation of the resulting estimates from the actual values. If the deviation is acceptable, the execution of the method of FIG. 3 may terminate. If the deviation is unacceptable, a retraining of the machine learning model may be performed starting at Step 308. Retraining may also involve updating of the hyperparameters of the machine learning model Alternatively, the retraining may be more comprehensive, and may involve the obtaining of new training data, starting at Step 302.

Turning to FIG. 4, a method 400 for predicting emissions events and/or to suggest mitigating actions, is shown. The method may be executed at any time, once the machine learning model has been trained using the method of FIG. 3.

In Step 402, current asset data are obtained, analogous to the obtaining of archived asset data in Step 302 of FIG. 3. However, unlike in Step 302, the majority of the current asset data are obtained from sensors, rather than from a database. Exceptions may include, for example, the historical asset data.

In Step 404, the current asset data are preprocessed, analogous to the preprocessing in Step 304 of FIG. 3.

In Step 406, the current asset data are standardized, analogous to the preprocessing in Step 306 of FIG. 3.

Steps 402-406 may be performed at regular intervals, e.g., every second, minute, hour, etc.

In Step 408, methane emissions events and/or mitigation actions are predicted using the previously trained machine learning model operating on the current asset data. The prediction of a methane emissions event may include a classification performed between multiple categories of methane emissions events of different magnitude, as previously described, and one or more mitigation actions may be predicted as appropriate, given the circumstances of the predicted methane emissions event. The exact nature of the prediction depends on the training of the machine learning model. For example, if the machine learning model was trained including timing, quantification, and location of the emissions event, the prediction may also include such information.

In Step 410, the predicted methane emissions events and/or suggested mitigating actions are reported to the user in a user visualization. The user may use user controls to review the predicted methane emissions events and/or suggested mitigating actions. Alternatively, mitigating actions may be automatically initiated. Examples of user interfaces are provided below.

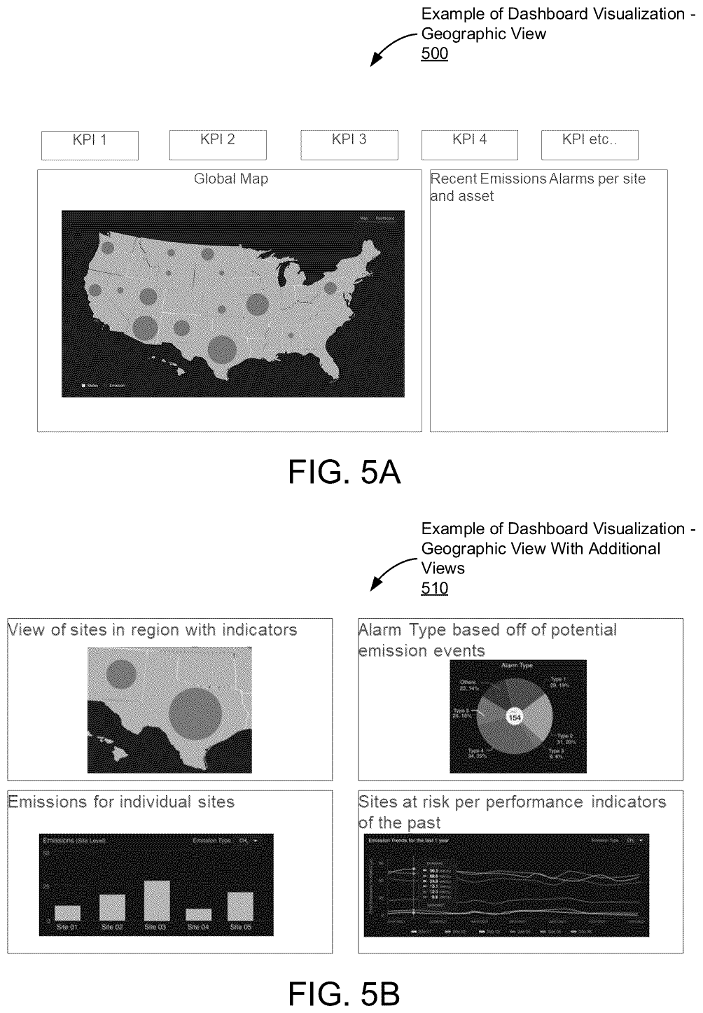

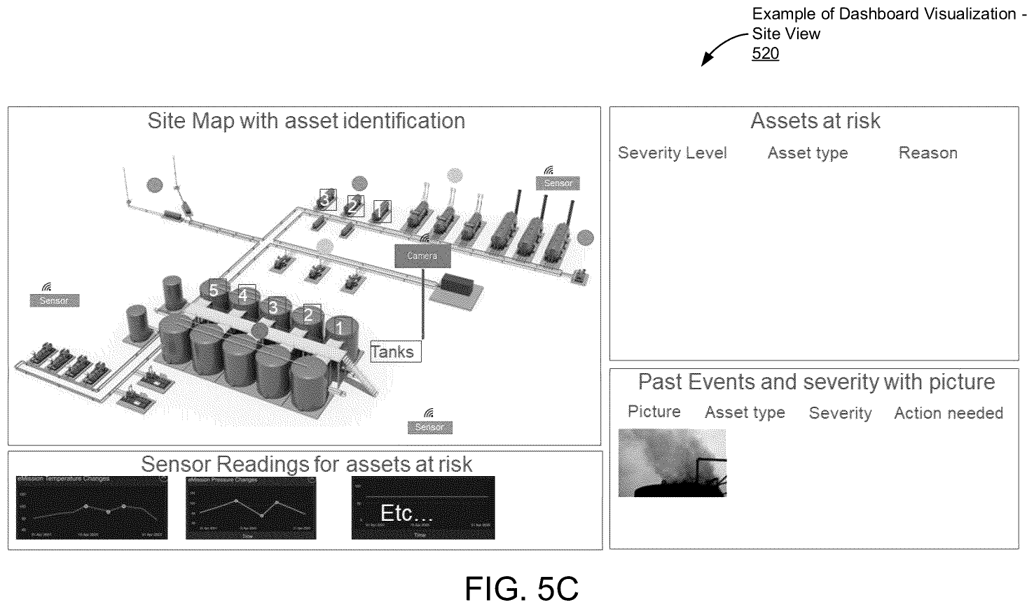

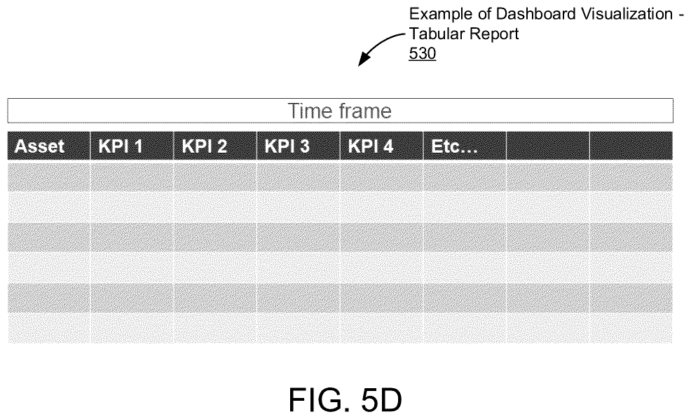

FIGS. 5A, 5B, 5C, and 5D show examples of dashboard visualizations, in accordance with one or more embodiments. As further discussed below, the dashboard visualizations are configurable to enable monitoring of methane emissions (predicted to occur in the future and/or past methane emissions) on a global, regional, side-wide, and asset-specific level.

The dashboard visualization 500 in FIG. 5A includes a global view and may be used to provide a quick overview of the locations of a deployed system. The size of a location may be indicated by scaling the symbol used to identify the location. Further a color coding of the symbol may be used to indicate the state (e.g., health) of the corresponding location. The dashboard visualization further includes a pane for recent emission alarms per site and/or per asset. For a more detailed evaluation, key performance indicators may be selected.

The dashboard visualization 510 in FIG. 5B includes four additional windows for user review at a regional level. A view of sites in a region provides a higher geographic resolution than the global view in the dashboard visualization 500. For the individual sites, emissions are displayed. A diagram that shows emissions events, organized by type, is further provided e.g. to enable rapid identify identification of frequently occurring types of emissions events. Finally, the dashboard visualization 510 also includes emissions trends over time, for different sites.

The dashboard visualization 520 in FIG. 5C includes additional windows for user review at a site-specific level. A site map that identifies the various assets is shown. Sensor readings for assets determined to be at risk are shown. In addition, for the assets at risk, further details are provided. In the example, these details include a severity level, the type of the asset, and an identified reason for considering the asset as being at risk. Further, past events are documented, including the asset type(s) involved, severity, photographic documentation, and actions needed to address the events.

As the dashboard visualization 530 in FIG. 5D shows, tabular reports may also be created. A tabular report may be created for any time frame, for any type(s) of key performance indicators, for any asset.

At least some of the dashboard visualizations are interactive, e.g., enable a user to explore and inspect data, and further to customize the dashboard visualizations as need or desired.

Embodiments of the disclosure have various benefits. Embodiments of the disclosure use machine learning to predict methane emissions events, without requiring extensive analysis by an operator. The resulting insights are data driven, and unlike conventional frequency-based inspection that may miss intermittent emissions, embodiments of the disclosure have the capability to predict emissions events for any point in time. In addition, embodiments of the disclosure have the capability to identify the root cause of emissions events. Embodiments of the disclosure further integrate well with existing platforms such as a SCADA system, thereby providing additional value without requiring extensive modifications of the existing platform. Because embodiments of the disclosure are sensor agnostic, existing sensing infrastructure may be integrated.

Embodiments of the disclosure use a cloud computing platform with a remotely accessible dashboard to report possible methane emissions events and/or proposed mitigating actions. Reporting time is reduced, providing a user with more time to react to the provided information, e.g., by initiating mitigating actions. A user may directly control onsite equipment via an edge computing platform to take mitigating action. Alternatively, one or more mitigating actions may be automatically performed. An actual methane emissions event may thus be avoided by taking action in response to the prediction. This may have the additional benefit of reducing downtime and increasing production time.

Embodiments of the disclosure may be provided to a customer (e.g., a petrochemical plant operator) in different forms. For example, software and hardware components (including sensors) may be provided. Alternatively, a solution that ties into existing sensors may be provided. The methods as described may be accessible in the form of a subscription with tiered service. For example, in a basic package, predicted methane emissions events are reported, including time data, and volume/trend of occurrence. In the next tier, the root cause data may be provided. In an additional tier, corrective actions may be reported and/or even implemented.

Actually performed studies have demonstrated the effectiveness of embodiments of the disclosure. For example, it was demonstrated that in an implementation that uses a digital twin to compute the Reid vapor pressure and automatically optimizes set points, flaring was cut by 60%, oil production was increased, and emissions were drastically reduced.

While embodiments of the disclosure have been described in the context of predicting and reducing/avoiding methane emissions events, embodiments of the disclosure may also be used for other purposes. For example, applications exist in the monitoring of landfills, and other markets besides oil and gas. Generally speaking, any type of emission may be monitored, including but not limited to CO₂, hydrogen, etc.

Embodiments may be implemented on a computer system. FIG. 6 is a block diagram of a computer system 602 used to provide computational functionalities associated with described algorithms, methods, functions, processes, flows, and procedures as described in the instant disclosure, according to an implementation. The illustrated computer 602 is intended to encompass any computing device such as a high performance computing (HPC) device, a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer 602 may include a computer that includes an input device, such as a keypad, keyboard, touch screen, or other device that can accept user information, and an output device that conveys information associated with the operation of the computer 602, including digital data, visual, or audio information (or a combination of information), or a GUI.

The computer 602 can serve in a role as a client, network component, a server, a database or other persistency, or any other component (or a combination of roles) of a computer system for performing the subject matter described in the instant disclosure. The illustrated computer 602 is communicably coupled with a network 630. In some implementations, one or more components of the computer 602 may be configured to operate within environments, including cloud-computing-based, local, global, or other environment (or a combination of environments).

At a high level, the computer 602 is an electronic computing device operable to receive, transmit, process, store, or manage data and information associated with the described subject matter. According to some implementations, the computer 602 may also include or be communicably coupled with an application server, e-mail server, web server, caching server, streaming data server, business intelligence (BI) server, or other server (or a combination of servers).

The computer 602 can receive requests over network 630 from a client application (for example, executing on another computer 602) and responding to the received requests by processing the said requests in an appropriate software application. In addition, requests may also be sent to the computer 602 from internal users (for example, from a command console or by other appropriate access method), external or third-parties, other automated applications, as well as any other appropriate entities, individuals, systems, or computers.

Each of the components of the computer 602 can communicate using a system bus 603. In some implementations, any or all of the components of the computer 602, both hardware or software (or a combination of hardware and software), may interface with each other or the interface 604 (or a combination of both) over the system bus 603 using an application programming interface (API) 612 or a service layer 613 (or a combination of the API 612 and service layer 613). The API 612 may include specifications for routines, data structures, and object classes. The API 612 may be either computer-language independent or dependent and refer to a complete interface, a single function, or even a set of APIs. The service layer 613 provides software services to the computer 602 or other components (whether or not illustrated) that are communicably coupled to the computer 602. The functionality of the computer 602 may be accessible for all service consumers using this service layer. Software services, such as those provided by the service layer 613, provide reusable, defined business functionalities through a defined interface. For example, the interface may be software written in JAVA, C++, or other suitable language providing data in extensible markup language (XML) format or other suitable format. While illustrated as an integrated component of the computer 602, alternative implementations may illustrate the API 612 or the service layer 613 as stand-alone components in relation to other components of the computer 602 or other components (whether or not illustrated) that are communicably coupled to the computer 602. Moreover, any or all parts of the API 612 or the service layer 613 may be implemented as child or sub-modules of another software module, enterprise application, or hardware module without departing from the scope of this disclosure.

The computer 602 includes an interface 604. Although illustrated as a single interface 604 in FIG. 6, two or more interfaces 604 may be used according to particular needs, desires, or particular implementations of the computer 602. The interface 604 is used by the computer 602 for communicating with other systems in a distributed environment that are connected to the network 630. Generally, the interface 604 includes logic encoded in software or hardware (or a combination of software and hardware) and operable to communicate with the network 630. More specifically, the interface 604 may include software supporting one or more communication protocols associated with communications such that the network 630 or interface's hardware is operable to communicate physical signals within and outside of the illustrated computer 602.

The computer 602 includes at least one computer processor 605. Although illustrated as a single computer processor 605 in FIG. 6, two or more processors may be used according to particular needs, desires, or particular implementations of the computer 602. Generally, the computer processor 605 executes instructions and manipulates data to perform the operations of the computer 602 and any algorithms, methods, functions, processes, flows, and procedures as described in the instant disclosure.

The computer 602 also includes a memory 606 that holds data for the computer 602 or other components (or a combination of both) that can be connected to the network 630. For example, memory 606 can be a database storing data consistent with this disclosure. Although illustrated as a single memory 606 in FIG. 6, two or more memories may be used according to particular needs, desires, or particular implementations of the computer 602 and the described functionality. While memory 606 is illustrated as an integral component of the computer 602, in alternative implementations, memory 606 can be external to the computer 602.

The application 607 is an algorithmic software engine providing functionality according to particular needs, desires, or particular implementations of the computer 602, particularly with respect to functionality described in this disclosure. For example, application 607 can serve as one or more components, modules, applications, etc. Further, although illustrated as a single application 607, the application 607 may be implemented as multiple applications 607 on the computer 602. In addition, although illustrated as integral to the computer 602, in alternative implementations, the application 607 can be external to the computer 602.

There may be any number of computers 602 associated with, or external to, a computer system containing computer 602, each computer 602 communicating over network 630. Further, the term “client,” “user,” and other appropriate terminology may be used interchangeably as appropriate without departing from the scope of this disclosure. Moreover, this disclosure contemplates that many users may use one computer 602, or that one user may use multiple computers 602.

In some embodiments, the computer 602 is implemented as part of a cloud computing system. For example, a cloud computing system may include one or more remote servers along with various other cloud components, such as cloud storage units and edge servers. In particular, a cloud computing system may perform one or more computing operations without direct active management by a user device or local computer system. As such, a cloud computing system may have different functions distributed over multiple locations from a central server, which may be performed using one or more Internet connections. More specifically, cloud computing system may operate according to one or more service models, such as infrastructure as a service (IaaS), platform as a service (PaaS), software as a service (SaaS), mobile “backend” as a service (MBaaS), serverless computing, artificial intelligence (AI) as a service (AlaaS), and/or function as a service (FaaS).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A method, comprising:
obtaining current asset data for an asset, the current asset data comprising process data;
predicting, using a machine learning model, a methane emissions event associated with the asset, based on the current asset data; and
reporting the predicted methane emissions event in a user visualization.

2. The method of claim 1, wherein the asset comprises at least one petrochemical asset.

3. The method of claim 1, wherein the current asset data further comprises at least one selected from a group consisting of:
environmental data,
historical data associated with the asset, and
methane sensor data.

4. The method of claim 1, wherein the prediction of the methane emissions event comprises a classification performed between multiple categories of methane emissions events of different magnitude.

5. The method of claim 1, wherein the prediction of the methane emissions event comprises a prediction of at least one selected from a group consisting of a timing, a location, and a quantification of the methane emissions event.

6. The method of claim 1, further comprising:
predicting, using the machine learning model, a mitigation action for the methane emissions event.

7. The method of claim 6, wherein the mitigation action comprises adjusting a setting of a valve associated with the asset.

8. The method of claim 6, further comprising:
performing the mitigation action such that an actual occurrence of the predicted methane emissions event is avoided.

9. The method of claim 1, further comprising, prior to performing the prediction:
obtaining, for the asset, archived asset data comprising process data and methane sensor data; and
training the machine learning model to predict methane emissions events based on the archived asset data used as training data.

10. The method of claim 9, further comprising, prior to training the machine learning model:
preprocessing the archived asset data, comprising at least one selected from a group consisting of removing outliers and removing false positives.

11. The method of claim 9, further comprising, prior to training the machine learning model:
standardizing the archived asset data for sensor-agnostic operation of the machine learning model.

12. A system, comprising:
a computing environment that:
obtains current asset data for an asset, the current asset data comprising process data,
predicts, using a machine learning model, a methane emissions event associated with the asset, based on the current asset data; and
a dashboard comprising a user visualization that reports the predicted methane emissions event.

13. The system of claim 12, wherein the machine learning model is a digital twin that establishes a virtual model that reflects characteristics of a physical environment related to the methane emissions event.

14. The system of claim 13,
wherein the asset is in the physical environment reflected by the virtual model, and
wherein the asset is one selected from a group consisting of a vapor recovery unit, a compressor, storage tank, a power unit, a valve, a flange, and a seal.

15. The system of claim 14, wherein the physical environment comprises sensors that obtain the current asset data for the asset in the physical environment.

16. The system of claim 15, wherein the sensors comprise at least one selected from a group consisting of a fenceline sensor, a thermal camera, a non-thermal camera, an optical gas imaging camera, a drone-based sensor, a robot-based sensor, a helicopter-based sensor, an airplane-based sensor, and a satellite-based sensor.

17. The system of claim 15,
wherein the computing environment comprises an edge computing platform that receives the current asset data from the sensors, and forwards the current asset data to the digital twin.

18. The system of claim 13,
wherein the computing environment comprises a cloud computing platform, and
wherein the digital twin is executed on the cloud computing platform.

19. The system of claim 13,
wherein the computing environment comprises a supervisor control and data acquisition (SCADA) system that obtains the process data associated with the asset and forwards the process data to the digital twin.

20. The system of claim 12,
wherein the user visualization in the dashboard is configurable to enable monitoring of the methane emissions on a global, regional, side-wide, and asset-specific level.
